(19) United States
(12) Patent Application Publication
(10) Pub. No.: US 2012/0203109 A1
(43) Pub. Date: Aug. 9, 2012

(54) ULTRASOUND DIAGNOSTIC APPARATUS AND ULTRASOUND IMAGE PRODUCING METHOD

(75) Inventors: Tsuyoshi TANABE, Ashigara-kami-gun (JP); Kimito KATSUYAMA, Ashigara-kami-gun (JP)

(73) Assignee: FUJIFILM CORPORATION, Tokyo (JP)

(21) Appl. No.: 13/361,449

(22) Filed: Jan. 30, 2012

(30) Foreign Application Priority Data
Feb. 9, 2011 (JP) 2011-025881
Feb. 9, 2011 (JP) 2011-025981
Feb. 9, 2011 (JP) 2011-026163

(51) Int. Cl. A61B 8/14

(52) U.S. Cl. 600/447; 600/443

(57) ABSTRACT

An ultrasound diagnostic apparatus includes: a controller for controlling a transmission circuit and a reception circuit to obtain reception data for measuring a sound speed and to obtain first reception data for producing a B mode image; a sound speed map producer for producing a sound speed map for the inside of the region of interest based on the reception data for measuring a sound speed; and an image producer for producing a B mode image based on the first reception data for producing a B mode image and second reception data for producing a B mode image, the second reception data for producing a B mode image being reception data for measuring a sound speed obtained by transmitting and receiving ultrasonic beams with forming transmission focuses at points having a depth closest to the given depth on sound rays passing inside the region of interest.

### BACKGROUND OF THE INVENTION

The present invention relates to an ultrasound diagnostic apparatus and an ultrasound image producing method and particularly to an ultrasound diagnostic apparatus that produces both a B mode image and a sound speed map for the inside of a region of interest by transmitting and receiving ultrasonic waves to and from a transducer array of an ultrasound probe.

Conventionally, ultrasound diagnostic apparatus using ultrasound images are employed in the medical field. In general, this type of ultrasound diagnostic apparatus comprises an ultrasound probe having a built-in transducer array and an apparatus body connected to the ultrasound probe. The ultrasound probe transmits an ultrasonic beam toward the inside of a subject's body, receives ultrasonic echoes from the subject, and the apparatus body electrically processes the reception signals to produce an ultrasound image.

In recent years, the sound speed at a site under examination is measured to achieve examination of the site inside the subject's body with a higher accuracy.

JP 2010-99452 A, for example, proposes an ultrasound diagnostic apparatus whereby a plurality of lattice points are set around a site under examination and an ultrasonic beam is transmitted to and received from the lattice points to obtain reception data, based on which local sound speeds are calculated.

JP 2010-99452 A describes a device having an ultrasound probe that transmits and receives an ultrasonic beam to and from the inside of a subject's body to obtain local sound speeds at a site under examination, thereby enabling display of a B mode image with the local sound speeds superimposed over it, for example.

When giving a diagnosis on a specific region inside a subject's body, displaying a sound speed map representing a distribution of local sound speeds at points in the region together with a B mode image signal is of advantage.

However, an attempt to produce both a B mode image and a sound speed map of a site under examination necessitates transmission and reception of an ultrasonic beam a number of times and requires much time and effort to obtain data for producing a B mode image and data for measuring the sound speed.

### SUMMARY OF THE INVENTION

An object of the present invention is to eliminate such problems associated with the prior art and provide an ultrasound diagnostic apparatus and an ultrasound image producing method capable of efficiently obtaining data for producing a B mode image and data for measuring the sound speed to achieve production of both a B mode image and a sound speed map.

An ultrasound diagnostic apparatus according to a first aspect of the present invention comprises:

an ultrasound probe including a transducer array;

a transmission circuit for supplying an actuation signal to the transducer array of the ultrasound probe to cause the transducer array to emit ultrasonic beams to a subject;

a reception circuit for processing reception signals outputted from the transducer array of the ultrasonic probe having received ultrasonic echoes from the subject;

a region-of-interest setting unit for setting a region of interest in an imaged region;

a controller for controlling the transmission circuit and the reception circuit to obtain reception data for measuring a sound speed by transmitting and receiving ultrasonic beams with forming transmission focuses at a plurality of points located in a predetermined pattern on sound rays passing inside the region of interest set by the region-of-interest setting unit, and to obtain first reception data for producing a B mode image by transmitting and receiving ultrasonic beams with forming a transmission focus at at least one given depth for sound rays passing outside the region of interest;

a sound speed map producer for producing a sound speed map for the inside of the region of interest based on the reception data for measuring a sound speed; and

an image producer for producing a B mode image based on the first reception data for producing a B mode image and second reception data for producing a B mode image, the second reception data for producing a B mode image being reception data for measuring a sound speed obtained by transmitting and receiving ultrasonic beams with forming transmission focuses at lattice points having a depth closest to the given depth among the plurality of points located in the predetermined pattern on the sound rays passing inside the region of interest.

An ultrasound diagnostic apparatus according to a second aspect of the present invention comprises:

an ultrasound probe including a transducer array;

a transmission circuit for supplying an actuation signal to the transducer array of the ultrasound probe to cause the transducer array to emit ultrasonic beams to a subject;

a reception circuit for processing reception signals outputted from the transducer array of the ultrasonic probe having received ultrasonic echoes from the subject;

a region-of-interest setting unit for setting a region of interest in an imaged region;

a controller for controlling the transmission circuit and the reception circuit to obtain reception data for measuring a sound speed by transmitting and receiving ultrasonic beams with forming transmission focuses at a plurality of points located in a predetermined pattern inside the region of interest set by the region-of-interest setting unit, and to obtain reception data for producing a B mode image by transmitting and receiving ultrasonic beams with forming a transmission focus at a given depth for each of sound rays;

a sound speed map producer for producing a sound speed map for the inside of the region of interest based on the reception data for measuring a sound speed; and

an image producer for producing a B mode image based on the reception data for producing a B mode image,

the controller controlling the transmission circuit and the reception circuit to obtain the reception data for producing a B mode image during production of the sound speed map by the sound speed map producer after obtaining the reception data for measuring a sound speed.

An ultrasound diagnostic apparatus according to a third aspect of the present invention comprises:

an ultrasound probe including a transducer array,

a transmission circuit for supplying an actuation signal to the transducer array of the ultrasound probe to cause the transducer array to emit ultrasonic beams to a subject,

a reception circuit for processing reception signals outputted from the transducer array of the ultrasonic probe having received ultrasonic echoes from the subject,

a region-of-interest setting unit for setting a region of interest in an imaged region,

a controller for controlling the transmission circuit and the reception circuit to obtain reception data for measuring a sound speed by transmitting and receiving ultrasonic beams with forming transmission focuses at a plurality of points located in a predetermined pattern inside the region of interest set by the region-of-interest setting unit, and to obtain reception data for producing a B mode image by transmitting and receiving an ultrasonic beam having a narrow width portion extending over a plurality of sound rays at a given depth for every two or more sound rays;

a sound speed map producer for producing a sound speed map for the inside of the region of interest based on the reception data for measuring a sound speed; and

an image producer for producing a B mode image based on the reception data for producing a B mode image,

A method of producing an ultrasound image according to a fourth aspect of the present invention comprises the steps of:

setting a region of interest in an imaged region and setting a plurality of points located in a predetermined pattern on sound rays passing inside the region of interest;

obtaining reception data for measuring a sound speed by transmitting and receiving ultrasonic beams with forming transmission focuses at the plurality of points;

producing a sound speed map for the inside of the region of interest based on the reception data for measuring a sound speed;

obtaining reception data for producing a B mode image by transmitting and receiving ultrasonic beams with forming transmission focuses at at least one given depth for sound rays passing outside the region of interest; and

producing a B mode image based on the first reception data for producing a B mode image and second reception data for producing a B mode image, the second reception data for producing a B mode image being reception data for measuring a sound speed obtained by transmitting and receiving ultrasonic beams with forming transmission focuses at points having a depth closest to the given depth among the plurality of points located in the predetermined pattern on the sound rays passing inside the region of interest.

A method of producing an ultrasound image according to a fifth aspect of the present invention comprises the steps of:

setting a region of interest in an imaged region and setting a plurality of points located in a predetermined pattern inside the region of interest;

obtaining reception data for measuring a sound speed by transmitting and receiving ultrasonic beams with forming transmission focuses at the plurality of points;

starting to produce a sound speed map for the inside of the region of interest based on the reception data for measuring a sound speed;

obtaining reception data for producing a B mode image by transmitting and receiving ultrasonic beams with forming transmission focuses at one given depth during producing of the sound speed map; and

producing a B mode image based on the obtained reception data for producing a B mode image.

A method of producing an ultrasound image according to a sixth aspect of the present invention comprises the steps of:

setting a region of interest in an imaged region and setting a plurality of points located in a predetermined pattern inside the region of interest;

obtaining reception data for measuring a sound speed by transmitting and receiving ultrasonic beams with forming transmission focuses at the plurality of points;

producing a sound speed map for the inside of the region of interest based on the reception data for measuring a sound speed;

obtaining reception data for producing a B mode image by transmitting and receiving an ultrasonic beam having a narrow width portion extending over a plurality of sound rays at a given depth for every two or more sound rays; and

producing a B mode image based on the reception data for producing a B mode image.

### BRIEF DESCRIPTION OF THE DRAWINGS

FIG. 1 is a block diagram illustrating a configuration of an ultrasound diagnostic apparatus according to Embodiment 1 of the invention.

FIGS. 2A and 2B schematically illustrate a principle of sound speed calculation according to Embodiment 1.

FIG. 3 illustrates transmission focuses for producing a B mode image and transmission focuses for measuring a sound speed according to Embodiment 1.

FIG. 4 illustrates transmission focuses for producing a B mode image and a transmission focuses for measuring a sound speed according to a variation of Embodiment 1.

FIG. 5 illustrates transmission focuses for producing a B mode image and transmission focuses for measuring a sound speed according to Embodiment 2.

FIG. 6 illustrates transmission focuses for producing a B mode image and transmission focuses for measuring a sound speed according to a variation of Embodiment 2.

FIG. 7 illustrates transmission focuses for producing a B mode image and transmission focuses for measuring a sound speed according to Embodiment 3.

FIG. 8 illustrates transmission focuses for producing a B mode image and transmission focuses for measuring a sound speed according to Embodiment 4.

FIG. 9 illustrates transmission focuses for producing a B mode image and transmission focuses for measuring a sound speed according to a variation of Embodiment 4.

FIG. 10 illustrates transmission focuses for producing a B mode image and transmission focuses for measuring a sound speed according to another variation of Embodiment 4.

FIG. 11 illustrates positions of transmission focuses and an order of transmission and reception of ultrasonic beams according to Embodiment 5.

FIG. 12 illustrates positions of transmission focuses and an order of transmission and reception of ultrasonic beams according to Embodiment 6.

FIG. 13 illustrates positions of transmission focuses and an order of transmission and reception of ultrasonic beams according to a variation of Embodiment 6.

FIG. 14 illustrates positions of transmission focuses and a state of an ultrasonic beam according to Embodiment 7.

FIG. 15 illustrates positions of transmission focuses and a state of an ultrasonic beam according to Embodiment 8.

FIG. 16 illustrates positions of transmission focuses and a state of an ultrasonic beam according to a variation of Embodiment 8.

### DETAILED DESCRIPTION OF THE INVENTION

Embodiments of the present invention will be described below based on the appended drawings.

### Embodiment 1

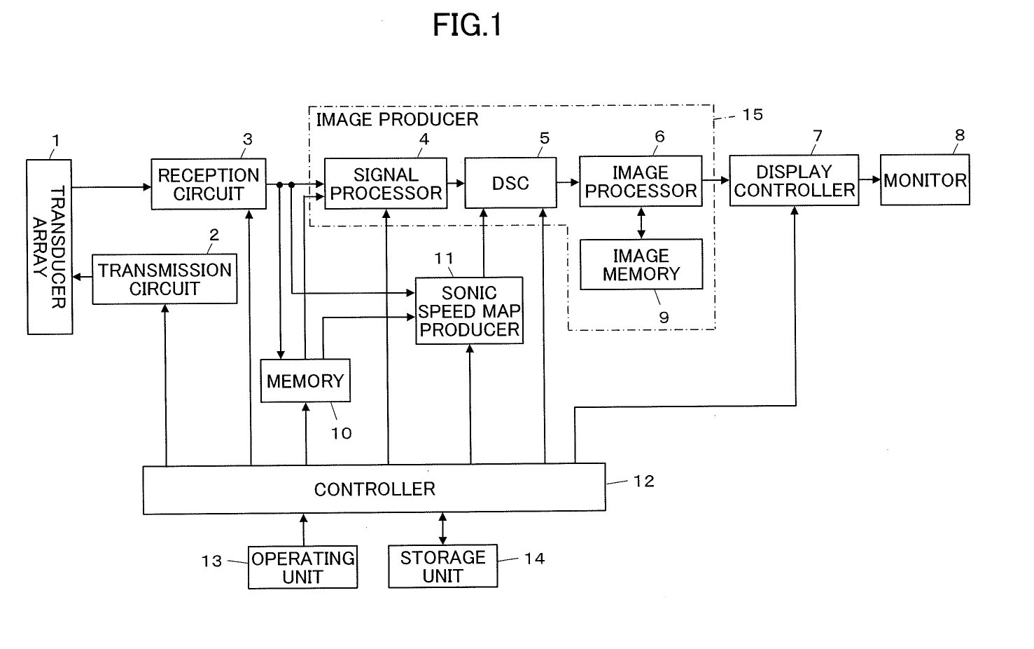

FIG. 1 illustrates a configuration of the ultrasound diagnostic apparatus according to Embodiment 1 of the invention. The ultrasound diagnostic apparatus comprises a transducer array 1, which is connected to a transmission circuit 2 and a reception circuit 3. The reception circuit 3 is connected to a signal processor 4, a DSC (Digital Scan Converter) 5, and an image processor 6 in sequence. The image processor 6 is connected via a display controller 7 to a monitor 8 and to an image memory 9.

The reception circuit 3 is connected to a memory 10 and a sound speed map producer 11. The transmission circuit 2, the reception circuit 3, the signal processor 4, the DSC 5, the display controller 7, the memory 10, and the sound speed map producer 11 are connected to a controller 12. The controller 12 is also connected to an operating unit 13 and a storage unit 14.

The transducer array 1 comprises a plurality of ultrasound transducers arranged one-dimensionally or two-dimensionally. These ultrasound transducers each transmit ultrasonic waves according to actuation signals supplied from the transmission circuit 2 and receive ultrasonic echoes from the subject to output reception signals. Each of the ultrasound transducers 11 comprises a oscillator composed of a piezoelectric body and electrodes each provided on both ends of the piezoelectric body. The piezoelectric body is composed of, for example, a piezoelectric ceramic represented by a PZT (titanate zirconate lead), a polymeric piezoelectric device represented by PVDF (polyvinylidene flouride), or a piezoelectric monochristal represented by PMN-PT (lead magnesium niobate lead titanate solid solution).

When the electrodes of each of the oscillators are supplied with a pulsed voltage or a continuous-wave voltage, the piezoelectric body expands and contracts to cause the oscillator to produce pulsed or continuous ultrasonic waves. These ultrasonic waves are combined to form an ultrasonic beam. Upon reception of propagating ultrasonic waves, each oscillator expands and contracts to produce an electric signal, which is then outputted as an ultrasonic reception signal.

The transmission circuit 2 includes, for example, a plurality of pulsers and adjusts the delay amounts for actuation signals based on a transmission delay pattern selected according to an instruction signal transmitted from the transmission controller 12 so that the ultrasonic waves transmitted from a plurality of ultrasound transducers of the transducer array 1 form an ultrasonic beam and supplies the ultrasound transducers with delay-adjusted actuation signals.

The reception circuit 3 amplifies and A/D-converts the reception signals transmitted from the ultrasound transducers of the transducer array 1, and then performs reception focusing processing by providing the reception signals with respective delays according to the sound speed or sound speed distribution that is set based on a reception delay pattern selected according to the control signal from the controller 12 and adding them up. This reception focusing processing yields reception data (sound ray signals) having the ultrasonic echoes well focused.

The signal processor 4 corrects attenuation of the reception data produced by the reception circuit 3, the attenuation depending on the distance that varies with the depth at which the ultrasonic waves are reflected, and then performs envelope detection processing to produce a B mode image signal, which is tomographic image information on a tissue inside the subject's body.

The DSC 5 converts the B mode image signal produced by the signal processor 4 into an image signal compatible with an ordinary television signal scanning mode (raster conversion).

The image processor 6 performs various processing required including gradation processing on the B mode image signal entered from the DSC 5 before outputting the B mode image signal to the display controller 7 or storing the B mode image signal in the image memory 9.

The signal processor 4, the DSC 5, the image processor 6, and the image memory 9 constitute the image producer 15 in the present invention.

The display controller 7 causes the monitor 8 to display an ultrasound diagnostic image according to the B mode image signal having undergone image processing by the image processor 6.

The monitor 8 includes a display device such as an LCD, for example, and displays an ultrasound diagnostic image under the control of the display controller 7.

The memory 10 sequentially stores the reception data outputted from the reception circuit 3. The memory 10 stores information on a frame rate entered from the controller 12 in association with the above reception data. Such information includes, for example, the depth of a position at which the ultrasonic wave is reflected, the density of scan lines, and a parameter representing the range of the visual field.

Under the control by the controller 12, the sound speed map producer 11 calculates the local sound speeds in a tissue inside the subject's body under examination based on the reception data stored in the memory 10 to produce the sound speed map.

The controller 12 controls the components in the ultrasound diagnostic apparatus according to the instruction entered by the operator using the operating unit 13.

The operating unit 13, provided for the operator to perform input operations, constitutes a region-of-interest setting unit and may be composed of, for example, a keyboard, a mouse, a track ball, and/or a touch panel.

The storage unit 14 stores, for example, an operation program and may be constituted by, for example, a recording medium such as an MO, an MT, a RAM, a CD-ROM, a DVD-ROM, an SD card, a CF card, or a USB memory, or a server.

The signal processor 4, the DSC 5, the image processor 6, the display controller 7, and the sound speed map producer 11 are each constituted by a CPU and an operation program for causing the CPU to perform various kinds of processing, but they may be each constituted by a digital circuit.

The operator may select one of the following three display modes. They are: a mode for displaying the B mode image alone, a mode for displaying the B mode image, with the sound speed map superimposed over it (e.g., display with color distinction or by varying luminance according to the local sound speed, or display where points having an equal local sound speed are connected by a line), and a mode for displaying the B mode image and the sound speed map image in juxtaposition. The B mode image may be displayed in a desired mode out of these modes.

When the B mode image is displayed, a plurality of ultrasound transducers of the transducer array 1 first transmit ultrasonic waves according to the actuation signals supplied from the transmission circuit 2, and the ultrasound transducers that have received ultrasonic echoes from the subject output the reception signals to the reception circuit 3, which produces the reception data. The signal processor 4, having received the reception data, produces the B mode image signal, the DSC 5 performs raster conversion of the B mode image signal, and the image processor 6 performs various image processing on the B mode image signal, whereupon, based on this B-mode image signal, the display controller 7 causes the monitor 8 to display the ultrasound diagnostic image.

The local sound speed may be calculated by a method described in JP 2010-99452 A filed by the Applicant of the present application.

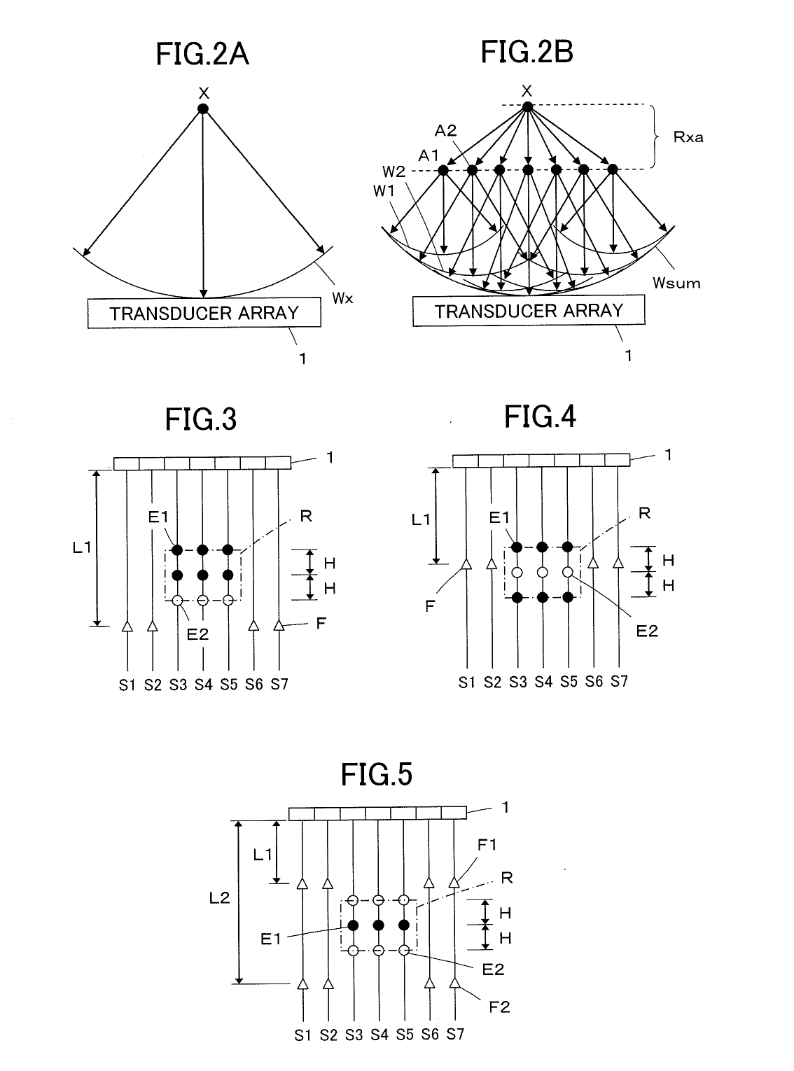

This method obtains the local sound speed at a lattice point X according to the Huygens principle. Suppose now that, on transmission of ultrasonic waves to the inside of a subject, a reception wave Wx reaches the transducer array 1 from the lattice point X, a reflection point in the subject, as illustrated in FIG. 2A, and that a plurality of lattice points A1, A2, . . . are arranged at equal intervals in positions shallower than the lattice point X, i.e., closer to the transducer array 1, as illustrated in FIG. 2B. Then, the local sound speed at the lattice point X is obtained according to the Huygens principle whereby a synthesized wave Wsum produced by combining reception waves W1, W2, . . . transmitted from the lattice points A1, A2, . . . having received a reception signal from the lattice point X coincides with the reception wave Wx from the lattice point X.

First, optimum sound speeds for all the lattice points X, A1, A2, . . . are obtained. The optimum sound speed herein means a sound speed allowing a highest image contrast and sharpness to be obtained as the set sound speed is varied after performing focus calculation on the lattice points based on the set sound speed and imaging to produce an ultrasound image. The optimum sound speed may be judged based on, for example, the image contrast, spatial frequency in the scan direction, and dispersion as described in JP 08-317926 A.

Next, the optimum sound speed for the lattice point X is used to calculate the waveform of an imaginary reception wave Wx emitted from the lattice point X.

Further, a hypothetical local sound speed V at the lattice point X is changed to various values to calculate the imaginary synthesized wave Wsum from the lattice points A1, A2, . . . Suppose that, at this time, the sound speed is consistent in a region Rxa between the lattice point X and the lattice points A1, A2, . . . and is equivalent to the local sound speed V at the lattice point X. The times in which the ultrasonic wave propagating from the lattice point X reaches the lattice points A1, A2, . . . are XA1/V, XA2/V, . . ., respectively. XA1, XA2, . . . are the distances between the lattice point X and the lattice points A1, A2, . . . Combining the reflected waves emitted with respective delays corresponding to the times XA1/V, XA2/V, . . . yields an imaginary synthesized wave Wsum.

Next, the respective differences between a plurality of the imaginary synthesized waves Wsum calculated by changing hypothetical local sound speed V at the lattice point X to various values and imaginary reception waves Wx from the lattice point X are calculated to determine a hypothetical local sound speed V at which the difference becomes a minimum as the local sound speed. The difference between the imaginary synthesized waves Wsum and the imaginary reception waves Wx from the lattice point X may be calculated by any of appropriate methods including a method using the cross-correlation, a method using phase matching addition by providing the reception waves Wx with a delay obtained from the synthesized wave Wsum, and a method using phase matching addition by providing the synthesized wave Wsum with a delay obtained from the reception signal Wx.

Thus, the local sound speeds inside a subject can be accurately calculated based on the reception data produced by the reception circuit 3. The sound speed map representing a distribution of the local sound speeds in a set region of interest may be likewise produced.

Now, referring to FIG. 3, transmission focuses for producing the B mode image and transmission focuses for measuring the sound speed according to Embodiment 1 will be described. For the sake of simplicity, FIG. 3 illustrates the transducer array 1 as having seven arrayed ultrasound transducers, showing how sound rays S1 to S7 are formed at intervals corresponding to those at which these ultrasound transducers are arrayed. In a region of interest R, lattice points E1 indicated by “” and lattice points E2 indicated by “◯” are set on sound rays passing through the region of interest R and located apart from each other by a distance of H in the depth direction. FIG. 3 shows a total of nine lattice points including six lattice points E1 and three lattice points E2 set on the sound rays S3 to S5 passing through the region of interest R. All these nine lattice points serve as transmission focuses to produce the sound speed map.

On the other hand, the transmission focuses for producing the B mode image are set on points F located on sound rays passing outside the region of interest R and located at a given depth. In FIG. 3, the points F are indicated by “Δ”. The transmission focus is set on a total of four points F located on sound rays S1, S2, S6, and S7 passing outside the region of interest R and located at a given depth of L1 among the sound rays S1 to S7. Forming the transmission focus at these four points F and transmitting and receiving an ultrasonic beam yield first reception data for producing the B mode image.

Transmission focuses used exclusively to produce the B mode image are not formed on the sound rays S3 to S5 passing through the region of interest R. Rather, the transmission focus is formed on three lattice points E2 located at a depth closest to the given depth Li among a plurality of lattice points set for measuring the sound speed, and the ultrasonic beam is transmitted and received to obtain reception data for measuring the sound speed, using the reception data thus obtained as second reception data for producing the B mode image.

Next, the operation of Embodiment 1 will be described.

First, according to the actuation signal from the transmission circuit 2, a plurality of ultrasound transducers of the transducer array 1 transmits an ultrasonic beam, and the ultrasound transducers that have received ultrasound echoes from a subject output reception signals to the reception circuit 3 to produce reception data, whereupon the display controller 7 causes the monitor 8 to display the B mode image based on the B mode image signal produced by the image producer 15.

When the operator operates the operating unit 13 to set the region of interest R in the B mode image displayed on the monitor 8, the controller 12 sets nine lattice points on the sound rays passing through the inside of the region of interest R, i.e., on the sound rays S3 to S5 so as to be apart from each other by intervals of H in the depth direction as illustrated in FIG. 3.

Next, the controller 12 sets a total of 4 points F on the sound rays S1, S2, S6, and S7 passing outside the region of interest R at the given depth L1. Three lattice points located at a depth closest to the given depth L1 among the nine lattice points set in the region of interest R become the lattice points E2 to serve as transmission focuses used for both the production of the B mode image and the measurement of the sound speed, whereas the remaining 6 lattice points become the lattice points E1 to serve as transmission focuses used exclusively for the measurement of the sound speed.

Then, the transmission focus is formed on each of the nine lattice points E1 and E2 inside the region of interest R and the 4 points F outside the region of interest R, and the controller 12 controls the transmission circuit 2 and the reception circuit 3 so that the ultrasonic beam is transmitted and received sequentially.

The reception data produced by the reception circuit 3 each time it receives the ultrasonic beam are outputted sequentially to the signal processor 4 of the image producer 15 and stored in the memory 10.

At this time, according to an instruction by the controller 12, the signal processor 4 uses reception data, among the reception data sequentially entered from the reception circuit 3, obtained by forming the transmission focus at four points F outside the region of interest R and three lattice points E2 inside the region of interest R to produce the B mode image signal. Specifically, the reception data for measuring the sound speed obtained by forming the transmission focus at the three lattice points E2 inside the region of interest R and transmitting and receiving the ultrasonic beam are used as second reception data for producing the B mode image, and the B mode image signal is produced based on the second reception data for producing the B mode image and the first reception data for producing the B mode image obtained by forming the transmission focus at the four points F outside the region of interest R and transmitting and receiving the ultrasonic beam. The B mode image signal is allowed to undergo raster conversion by the DSC 5 and various image processing by the image processor 6, sent to the display controller 7, and displayed on the monitor 8.

Upon receiving an instruction for production of the sound speed map, the sound speed map producer 11 uses reception data obtained by forming the transmission focus at the nine lattice points E1 and E2 inside the region of interest R and transmitting and receiving the ultrasonic beam among the reception data stored in the memory 10 to calculate the local sound speeds at the lattice points and produce the sound speed map for the inside of the region of interest R. The data on the sound speed map obtained by the sound speed map producer 11 are allowed to undergo raster conversion by the DSC 5 and various image processing by the image processor 6 before being transmitted to the display controller 7. Then, according to the display mode entered from the operating unit 13 by the operator, the sound speed map is displayed on the monitor 8 as superimposed over the B mode image, or the B mode image and the sound speed map are displayed in juxtaposition on the monitor 8.

Thus, by using the reception data for measuring the sound speed obtained by forming the transmission focus at the three lattice points E2 inside the region of interest R and transmitting and receiving the ultrasonic beam as second reception data for producing the B mode image, the reception data for producing the B mode image and the reception data for measuring the sound speed can be efficiently obtained to achieve both the production of the B mode image and the production of the sound speed map.

The ultrasonic beam that is transmitted with the transmission focus formed at the four points F outside the region of interest R exclusively to produce the B mode image and the ultrasonic beam that is transmitted with the transmission focus formed at nine lattice points E1 and E2 inside the region of interest R exclusively to measure the sound speed may differ from each other in, for example, central frequency and the number of apertures of the transducer array 1. For example, an ultrasonic beam having a central frequency of 3 MHz with 64 channels (number of apertures) may be used as ultrasonic beam exclusively used to produce the B mode image and an ultrasonic beam having a central frequency of 8 MHz with 96 channels (number of apertures) may be used as ultrasonic beam to measure the sound speed.

The ultrasonic beam for measuring the sound speed is preferably produced with broader apertures in order to narrow down the transmission focuses further and with a higher central frequency than for the ultrasonic beam for producing the B mode image in order to reduce the effects of side lobes. In cases where the beam is highly liable to refraction caused by, for example, the abdominal wall as when a liver is examined, the effects of refraction may be reduced by, conversely, lowering the central frequency of the ultrasonic beam for measuring the sound speed to, for example, 2.5 MHz.

In FIG. 3, the three lattice points E2 located at the deepest depth have a depth closest to the given depth L1 among the nine lattice points set inside the region of interest R, and these lattice points E2 serve as transmission focuses for both the production of the B mode image and the measurement of the sound speed, whereas when the three lattice points located at the center among the nine lattice points set in the region of interest R have a depth closest to the given depth L1 as illustrated in FIG. 4, these lattice points serve as lattice points E2 used for the both production of the B mode image and the measurement of the sound speed.

### Embodiment 2

While Embodiment 1 has one transmission focus at a given depth on each sound ray for transmission of the ultrasonic beam for producing the B mode image, a plurality of transmission focuses may be formed at a plurality of depths on the same sound ray. When, for example, a total of 4 points F1 located at the given depth L1 and a total of 4 points F2 located at a give depth L2 are set on the sound rays S1, S2, S6, and S7 passing outside the region of interest R as illustrated in FIG. 5, the transmission focus is formed at these points F1 and F2 to sequentially transmit and receive the ultrasonic beam.

In this case, the lattice points having respective depths closest to a plurality of given depths are selected as lattice points used for both the production of the B mode image and the measurement of the sound speed. In FIG. 5, among the nine lattice points set inside the region of interest R, a total of six lattice points, namely, three lattice points located in the shallowest position having a depth closest to the given depth L1 and three lattice points located in a deepest position having a depth closest to the given depth L2, become the lattice points E2 used for both the production of the B mode image and the measurement of the sound speed.

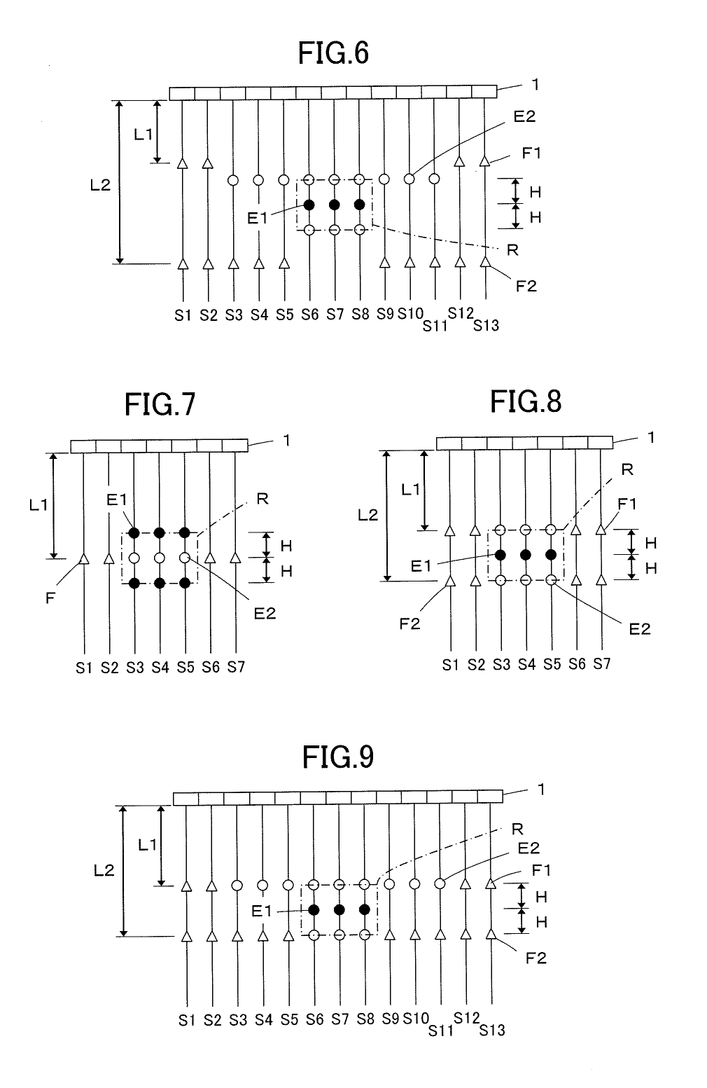

Because of the need to calculate the respective local sound speeds at the lattice points in the region of interest R, there are cases where lattice points are added outside the region of interest R, and the transmission focus for measuring the sound speed is formed also at these external lattice points as illustrated in FIG. 6. In this case, the lattice points set outside the region of interest R also become lattice points E2 used for both the production of the B mode image and the measurement of the sound speed, and the reception data obtained for measuring the sound speed are used as second reception data for producing the B mode image. FIG. 6 illustrates the sound rays S6 to S8 as passing through the region of interest R and the sound rays S1 to S5 and S9 to S13 as passing outside the region of interest R.

### Embodiment 3

While, in Embodiments 1 and 2, a plurality of lattice points are set inside the region of interest R, and the lattice points having a depth closest to a given depth at which the transmission focuses for an ultrasonic beam for producing the B mode image are formed are used for both the production of the B mode image and the measurement of the sound speed, the transmission focuses for producing the B mode image may, conversely, be so formed as to have a depth equal to that of the lattice points used for both the production of the B mode image and the measurement of the sound speed among the lattice points set inside the region of interest R.

As illustrated in FIG. 7, for example, nine lattice points are set inside the region of interest R, and among these lattice points, the three lattice points located at the center of the region of interest in the depth direction become lattice points E2 used for both the production of the B mode image and the measurement of the sound speed. In this case, a total of four points F are set on the sound rays S1, S2, S6, and S7 passing outside the region of interest R and having the same depth as the points E2, so that the transmission focus is formed on these points F the ultrasonic beam for producing the B mode image is transmitted and received.

The reception data for producing the B mode image and the reception data for measuring the sound speed can be efficiently obtained by the above method to achieve both the production of the B mode image and the measurement of the sound speed.

### Embodiment 4

In Embodiment 3, the transmission of the ultrasonic beam for producing the B mode image may be multistage focus transmission, so that a plurality of transmission focuses may be formed at a plurality of depths on the same sound ray. As illustrated in FIG. 8, for example, among the nine lattice points set inside the region of interest R, the three lattice points located in the shallowest position and the three lattice points located in the deepest position become lattice points E2 used for both the production of the B mode image and the measurement of the sound speed, whereas four points F1 having the same depth L1 as the lattice points E2 located at the shallowest depth and four points F2 having the same depth L2 as the lattice points E2 located in the deepest position are set on the sound rays S1 and S2 and S6 and S7, and the transmission focus is formed at these points F1 and F2 to perform transmission and reception of the ultrasonic beam for producing the B mode image.

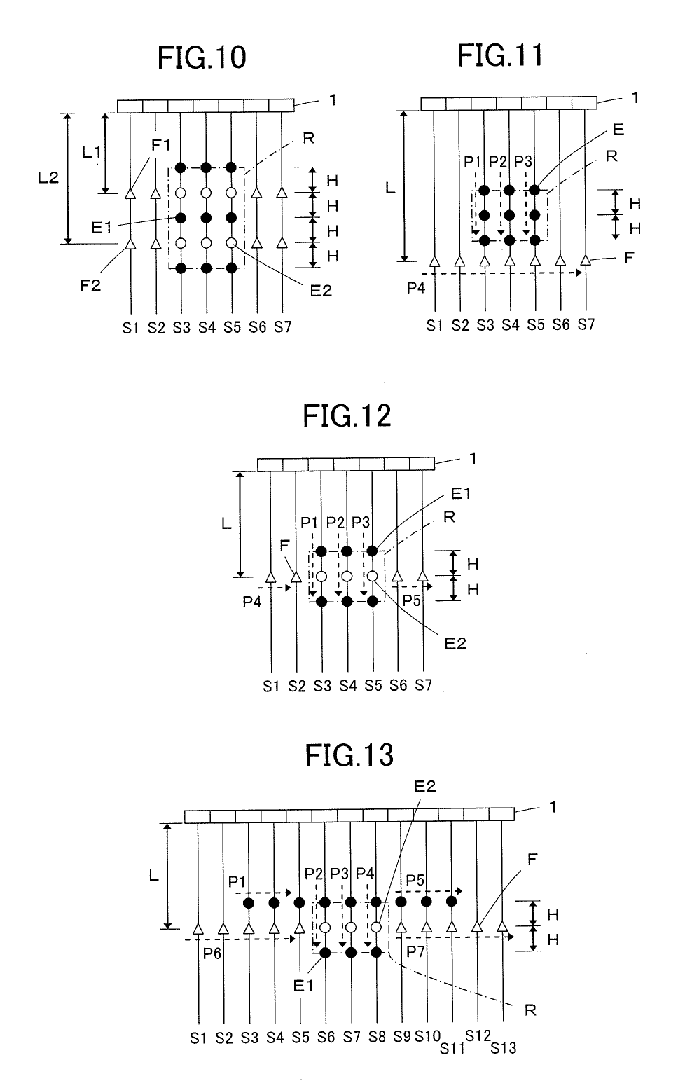

Because of the need to calculate the respective local sound speeds at the lattice points in the region of interest R, there are cases where lattice points are added outside the region of interest R, and the transmission focus for measuring the sound speed is formed also at these external lattice points as illustrated in FIG. 9. In this case, the lattice points set outside the region of interest R also become lattice points E2 used for both the production of the B mode image and the measurement of the sound speed, and the reception data obtained for measuring the sound speed are used as second reception data for producing the B mode image.

Where the region of interest R is longer in the depth direction as illustrated in FIG. 10, among the lattice points set inside the region of interest R, the lattice points located apart from the lattice points in the shallowest position and from the lattice points located in the deepest position inwardly of the region of interest R by a distance of, for example, one fourth of a length of the region of interest R in the depth direction may be used as lattice points E2 for both the production of the B mode image and the measurement of the sound speed.

### Embodiment 5

Referring now to FIG. 11, the transmission focuses for producing the B mode image and the transmission focuses for measuring the sound speed in Embodiment 5 will be described. For the sake of simplicity, FIG. 11 illustrates the transducer array 1 as having seven arrayed ultrasound transducers, showing how the sound rays S1 to S7 are formed at intervals corresponding to those at which these ultrasound transducers are arrayed. In the region of interest R, the lattice points E indicated by “” are set on the sound rays passing through the region of interest R and located apart from each other by a distance of H in the depth direction. FIG. 11 shows nine lattice points E set on the sound rays S3 to S5 passing through the region of interest R. All these nine lattice points E become transmission focuses to produce the sound speed map.

On the other hand, the transmission focuses for producing the B mode image are set at points F located on the sound rays S1 to S7 at a given depth. In FIG. 11, the points F are indicated by “Δ” and the transmission focus is formed at the seven points F located on the sound rays S1 to S7 and having a given depth L.

The transmission focus is formed at the nine lattice points E and the seven lattice points F set as described above to perform transmission and reception of the ultrasonic beam and obtain the reception data. In Embodiment 5, the transmission focus is first formed at the nine lattice points E in the region of interest R to perform transmission and reception of the ultrasonic beam and obtain the reception data for measuring the sound speed, and during calculation of the local sound speed and production of the sound speed map based on the obtained reception data for measuring the sound speed, the transmission focus is formed at seven points F to perform transmission and reception of the ultrasonic beam and obtain the reception data for producing the B mode image.

Next, the operation of Embodiment 5 will be described.

First, according to the actuation signal from the transmission circuit 2, a plurality of ultrasound transducers of the transducer array 1 transmits an ultrasonic beam, and the ultrasound transducers that have received ultrasonic echoes from a subject output reception signals to the reception circuit 3 to produce reception data, whereupon the display controller 7 causes the monitor 8 to display the B mode image based on the B mode image signal produced by the image producer 15.

When the operator operates the operating unit 13 to set the region of interest R in the B mode image displayed on the monitor 8, the controller 12 sets nine lattice points on the sound rays passing through the inside of the region of interest R, i.e., on the sound rays S3 to S5 illustrated in FIG. 11 so as to be apart from each other by an interval of H in the depth direction.

Next, the controller 12 sets a total of 7 points F that are located at the given depth L on the sound rays S1 to S7.

When the reception data have been obtained by transmitting and receiving the ultrasonic beam with the transmission focus formed at the nine lattice points E inside the region of interest R, the controller 12 controls the transmission circuit 2 and the reception circuit 3 so as to obtain the reception data for producing the B mode image by transmitting and receiving the ultrasonic beam with the transmission focus formed at the seven lattice points F.

Now, as illustrated in FIG. 11, for example, the transmission focus is sequentially formed at three lattice points E set on the sound ray S3 among the nine lattice points E inside the region of interest R to transmit and receive the ultrasonic beam in step P1, then the transmission focus is sequentially formed at three lattice points E set on the sound ray S4 to transmit and receive the ultrasonic beam in the next step P2, and further the transmission focus is sequentially formed at three lattice points E set on the sound ray S5 to transmit and receive the ultrasonic beam in step P3. Thus, the reception data for measuring the sound speed are obtained.

The reception data for measuring the sound speed are sequentially outputted from the reception circuit 3 to the memory 10 and stored therein. When all the reception data for the nine lattice points E have been obtained and stored in the memory 10, the sound speed map producer 11 starts calculation of the local sound speeds at the lattice points E and production of the sound speed map for the inside of the region of interest R using the reception data for measuring the sound speed stored in the memory 10 according to an instruction by the controller 12.

During the calculation of the local sound speeds and production of the sound speed map by the sound speed map producer 11, the transmission focus is formed now at the seven points F on the sound rays sequentially from S1 to S7 to transmit and receive the ultrasonic beam in step P4. Thus, the reception data for producing the B mode image are obtained.

The obtained reception data for producing the B mode image are sequentially outputted to the signal processor 4 of the image producer 15 and stored in the memory 10. The signal processor 4 uses the reception data for the B mode image sequentially entered from the reception circuit 3 to produce the B mode image signal, which undergoes raster conversion followed by various image processing through the image processor 6 and is transmitted to the display controller 7.

Upon production of the sound speed map for the region of interest R by the sound speed map producer 11, the sound speed map data are allowed to undergo raster conversion by the DSC 5 and various image processing by the image processor 6 before being transmitted to the display controller 7. Then, according to the display mode entered from the operating unit 13 by the operator, the B mode image is displayed, with the sound speed map superimposed over it, on the monitor 8, or the B mode image and the sound speed map are displayed in juxtaposition on the monitor 8.

Because, as described above, the reception data for measuring the sound speed are preferentially obtained, and the reception data for producing the B mode image are obtained while the sound speed map producer 11 is producing the sound speed map for the inside of the region of interest R based on the obtained reception data for measuring the sound speed, the data for producing the B mode image and the data for measuring the sound speed can be efficiently obtained to enable both the production of the B mode image and the production of the sound speed map.

In Embodiment 5, among the nine lattice points E inside the region of interest R, the transmission focus is sequentially formed at three lattice points E set on the sound ray S3 in step P1, then the transmission focus is sequentially formed at three lattice points E set on the sound ray S4 in the next step P2, and the transmission focus is sequentially formed at three lattice points E set on the sound ray S5 in step P3, thereby to transmit and receive the ultrasonic beam in each of these steps. However, the invention is not limited this way. Transmission and reception of the ultrasonic beam may be performed in such order that the transmission focus is sequentially formed at three lattice points E located at the same depth on the sound rays S3 to S5 first, and subsequently, the transmission focus is sequentially formed at three lattice points E located at another depth on the sound rays S3 to S5.

### Embodiment 6

While the seven points F serving as transmission focuses for producing the B mode image are set without regard to the nine lattice points E inside the region of interest R in Embodiment 5, the controller 12 may so control the transmission circuit 2 and the reception circuit 3 as to obtain the reception data used exclusively for producing the B mode image only for the sound rays passing outside the region of interest R whereas for the sound rays passing through the region of interest R, among the lattice points inside the region of interest R, the transmission focus may be formed at lattice points having a depth closest to the given depth L at which the transmission focus for producing the B mode image is formed to transmit and receive the ultrasonic beam in order to obtain the reception data for measuring the sound speed, which then is used as reception data for producing the B mode image in order for the image producer 15 to produce the B mode image.

In this case, the given depth L at which the transmission focus for producing the B mode image is formed may be so set as to be equal to the depth of any one of the lattice points set inside the region of interest R.

As illustrated in FIG. 12, for example, nine lattice points are set inside the region of interest R, and among these lattice points, a total of six lattice points located in the shallowest position and the deepest position become lattice points E1 used exclusively for measuring the sound speed whereas three lattice points located at the center of the region of interest R in the depth direction become lattice points E2 for both the production of the B mode image and the measurement of the sound speed. In FIG. 4, the lattice points E1 are indicated by “”, and the lattice points E2 are indicated by “◯”.

A total of four points F are set on the sound rays S1, S2, S6, and S7 passing outside the region of interest R and at the depth L equal to the depth of the lattice points E2.

Subsequently, the transmission focus is sequentially formed at three lattice points set on the sound ray S3 among the nine lattice points E1 and E2 inside the region of interest R to transmit and receive the ultrasonic beam in step P1, the transmission focus is sequentially formed at three lattice points set on the sound ray S4 to transmit and receive the ultrasonic beam in the next step P2, and the transmission focus is sequentially formed at three lattice points set on the sound ray S5 to transmit and receive the ultrasonic beam in step P3. Thus, the reception data for measuring the sound speed are obtained similarly to Embodiment 1.

The reception data for measuring the sound speed are sequentially outputted from the reception circuit 3 to the signal processor 4 and also to the memory 10 for storage, so that the sound speed map producer 11 uses the reception data for measuring the sound speed stored in the memory 10 to start calculation of the local sound speeds at the lattice points E1 and E2 and production of the sound speed map for the inside of the region of interest R.

During the calculation of the local sound speeds and production of the sound speed map by the sound speed map producer 11, the transmission focus is sequentially formed now at the points F on the sound rays S1 and S2 out of the four points F to transmit and receive the ultrasonic beam in step P4, and then the transmission focus is sequentially formed on the points F on the sound rays S6 and S7 to transmit and receive the ultrasonic beam in step P 5.

According to an instruction by the controller 12, the signal processor 4 uses reception data obtained by forming the transmission focus at three points E2 inside the region of interest R and by transmitting and receiving the ultrasonic beam and reception data obtained by forming the transmission focus at four points F outside the region of interest R and by transmitting and receiving the ultrasonic beam, among the reception data sequentially entered from the reception circuit 3, thereby to produce the B mode image signal. Specifically, the reception data for measuring the sound speed obtained by forming the transmission focus at the three lattice points E2 inside the region of interest R and transmitting and receiving the ultrasonic beam are used as second reception data for producing the B mode image for the sound rays S3 to S5 and, based on the reception data for producing the B mode image and the reception data for producing the B mode image for the sound rays S1, S2, S6, and S7 obtained by forming the transmission focus at four points F and transmitting and receiving the ultrasonic beam, the B mode image signal is produced. The B mode image signal undergoes raster conversion by the DSC 5 and various image processing by the image processor 6 and is sent to the display controller 7.

Upon production of the sound speed map for the region of interest R by the sound speed map producer 11, the sound speed map data are allowed to undergo raster conversion by the DSC 5 and various image processing by the image processor 6 before being transmitted to the display controller 7. Then, according to the display mode entered from the operating unit 13 by the operator, the B mode image is displayed, with the sound speed map superimposed over it, on the monitor 8, or the B mode image and the sound speed map are displayed in juxtaposition on the monitor 8.

In Embodiment 6, by using the reception data for measuring the sound speed obtained by transmitting and receiving the ultrasonic beam with the transmission focus formed at the three lattice points E2 inside the region of interest R also as reception data for producing the B mode image, the reception data for producing the B mode image and the reception data for measuring the sound speed can be more efficiently obtained to achieve both the production of the B mode image and the production of the sound speed map.

Because of the need to calculate the respective local sound speeds at the lattice points in the region of interest R, there are cases where lattice points are added outside the region of interest R, and the transmission focus for measuring the sound speed is formed also at these external lattice points as illustrated in FIG. 13. Also in this case, the reception data for measuring the sound speed may be preferentially obtained, and the reception data for producing the B mode image may be obtained while the sound speed map producer 11 is producing the sound speed map.

In FIG. 13, among the sound rays S1 to S13, the sound rays S6 to S8 are shown as passing through the region of interest R and the sound rays S1 to S5 and S9 to S13 are shown as passing outside the region of interest R, and the lattice points E1 are set also on the sound rays S3 to S5 and S9 to S11.

A total of ten points F are set on the sound rays S1 to S5 and S9 to S13 passing outside the region of interest R and at the depth L equal to the depth of the lattice points E2.

The transmission focus is sequentially formed at lattice points E1 on the sound rays S3 to S5 to transmit and receive the ultrasonic beam in step P1, the transmission focus is sequentially formed at three lattice points set on the sound ray S6 to transmit and receive the ultrasonic beam in step P2, the transmission focus is sequentially formed at three lattice points set on the sound ray S7 to transmit and receive the ultrasonic beam in step P3, the transmission focus is sequentially formed at three lattice points set on the sound ray S8 to transmit and receive the ultrasonic beam in step P4, and the transmission focus is sequentially formed at lattice points E1 on the sound rays S9 to S11 to transmit and receive the ultrasonic beam in step P5. Thus, the reception data for measuring the sound speed are obtained.

During the calculation of the local sound speeds and production of the sound speed map by the sound speed map producer 11, the transmission focus is sequentially formed at the points F on the sound rays S1 to S5 and the ultrasonic beam is transmitted and received in step P6, whereupon the transmission focus is sequentially formed on the sound rays S9 to S13, and the ultrasonic beam is transmitted and received in step P 7.

The reception data for measuring the sound speed obtained by transmitting and receiving the ultrasonic beam with the transmission focus formed at the three lattice points E2 in the region of interest R are used as reception data for producing the B mode image for the sound rays S6 to S8, and the B mode image signal is produced based on the reception data for producing the B mode image and the reception data for producing the B mode image for the sound rays S1 to S5 obtained by transmitting and receiving the ultrasonic beam with the transmission focus formed at ten points F.

### Embodiment 7

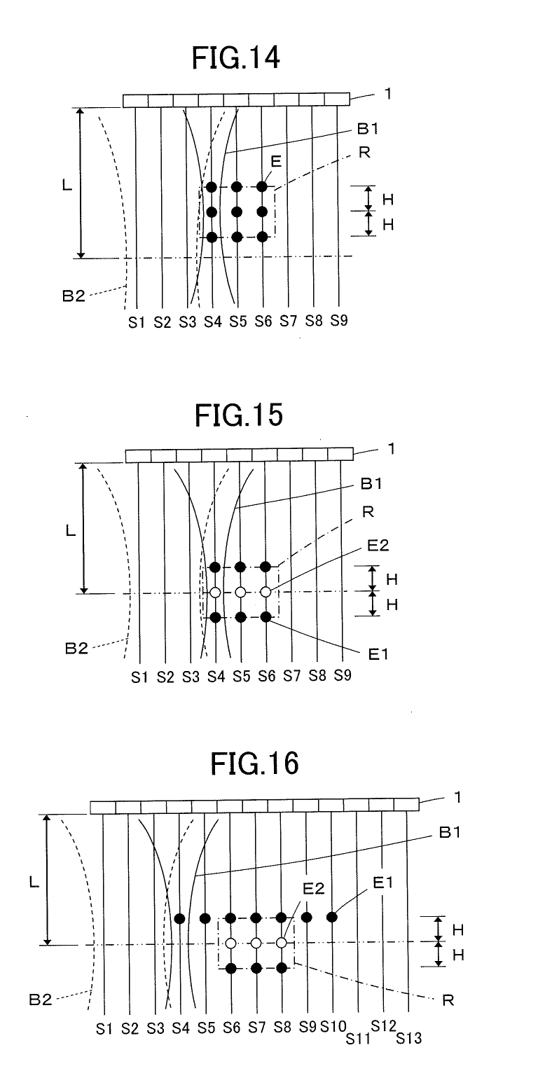

Referring to FIG. 14, the transmission focuses and the ultrasonic beam for measuring the sound speed and the ultrasonic beam for producing the B mode image in Embodiment 7 will be described. For simplicity, FIG. 14 illustrates the transducer array 1 as having nine arrayed ultrasound transducers, showing how sound rays S1 to S9 are formed at intervals corresponding to the array pitch of these ultrasound transducers. In the region of interest R, the lattice points E indicated by “” are set on the sound rays passing through the region of interest R and located apart from each other by a distance of H in the depth direction. FIG. 14 shows nine lattice points E set on the sound rays S4 to S6 passing through the region of interest R. All these nine lattice points E serve as transmission focuses to produce the sound speed map.

The reception data for measuring the sound speed are obtained by transmitting and receiving the ultrasonic beam so that the transmission focus is formed at the nine lattice points E set inside the region of interest R.

On the other hand, an ultrasonic beam B2 having a narrow width portion at the given depth L is used to produce the B mode image. The narrow width portion of the ultrasonic beam B2 has a width sufficient to extend over a plurality of sound rays. FIG. 14 shows the ultrasonic beam B2 having a narrow width portion extending over three sound rays S1 to S3.

The use of such wide ultrasonic beam B2 enables acquisition of reception data for producing the B mode image for three sound rays by transmitting and receiving the ultrasonic beam B2 only once. Thus, the ultrasonic beam B2 need not be transmitted and received for each of the sound rays and need only be transmitted and received for every three sound rays.

Next, the operation of Embodiment 7 will be described. First, according to the actuation signal from the transmission circuit 2, a plurality of ultrasound transducers of the transducer array 1 transmit an ultrasonic beam, and the ultrasound transducers that have received ultrasound echoes from a subject output reception signals to the reception circuit 3 to produce reception data, whereupon the display controller 7 causes the monitor 8 to display the B mode image based on the B mode image signal produced by the image producer 15.

When the operator operates the operating unit 13 to set the region of interest R in the B mode image displayed on the monitor 8, the controller 12 sets nine lattice points on the sound rays passing through the inside of the region of interest R, i.e., on the sound rays S4 to S6 in FIG. 14 so as to be apart from each other by a distance of H in the depth direction.

The controller 12 controls the transmission circuit 2 and the reception circuit 3 so as to obtain the reception data for measuring the sound speed by transmitting and receiving the ultrasonic beam with the transmission focus formed at the nine lattice points E in the region of interest R.

The obtained reception data for measuring the sound speed are sequentially outputted from the reception circuit 3 to the memory 10 and stored therein. When all the reception data for the nine lattice points E have been obtained and stored in the memory 10, the sound speed map producer 11 calculates the local sound speeds at the lattice points E and produces the sound speed map for the inside of the region of interest R using the reception data for measuring the sound speed stored in the memory 10 according to an instruction by the controller 12.

Further, the controller 12 controls the transmission circuit 2 and the reception circuit 3 so that a wide ultrasonic beam B2 having the narrow width portion at the given depth L may be transmitted and received. The narrow width portion of the ultrasonic beam B2 has a width extending over three sound rays adjacent to each other. First, the ultrasonic beam B2 having a narrow width portion extending over three sound rays S1 to S3 at the given depth L is transmitted to obtain reception data for producing the B mode image for the sound rays S1 to S3. Next, the ultrasonic beam B2 having a narrow width portion extending over three sound rays S4 to S6 at the given depth L is transmitted to obtain reception data for producing the B mode image for the sound rays S4 to S6. Further, the ultrasonic beam B2 having a narrow width portion extending over three sound rays S7 to S9 at the given depth L is transmitted to obtain reception data for producing the B mode image for the sound rays S7 to S9.

The use of such wide ultrasonic beam B2 enables acquisition of reception data for producing the B mode image for nine sound rays S1 to S9 by transmitting and receiving the ultrasonic beam B2 three times.

The thus produced reception data for producing the B mode image are sequentially outputted from the reception circuit 3 to the signal processor 4 of the image producer 15 and stored in the memory 10. The signal processor 4 uses the reception data for the B mode image sequentially entered from the reception circuit 3 to produce the B mode image signal, which undergoes raster conversion followed by various image processing through the image processor 6 and is transmitted to the display controller 7.

Upon production of the sound speed map for the region of interest R by the sound speed map producer 11, the sound speed map data are allowed to undergo raster conversion by the DSC 5 and various image processing by the image processor 6 before being transmitted to the display controller 7. Then, according to the display mode entered from the operating unit 13 by the operator, the B mode image is displayed, with the sound speed map superimposed over it, on the monitor 8, or the B mode image and the sound speed map are displayed in juxtaposition on the monitor 8.

Because the reception data for measuring the sound speed are obtained by forming the transmission focus at a plurality of lattice points E set in the region of interest R and by transmitting and receiving the ultrasonic beam B1 whereas the reception data for producing the B mode image are obtained by transmitting and receiving the wide ultrasonic beam B2 having a narrow width portion extending over a plurality of sound rays at the given depth L for every two or more sound rays, data for producing the B mode image and data for measuring the sound speed can be efficiently obtained, enabling both the production of the B mode image and the measurement of the sound speed.

While the wide ultrasonic beam B2 for producing the B mode image has the narrow width portion extending over three sound rays at the given depth L in Embodiment 7, the invention is not limited to this. An ultrasonic beam having a narrow width portion extending over a plurality of sound rays at the given depth L may be transmitted and received for every two or more sound rays to obtain the reception data for producing the B mode image.

### Embodiment 8

In Embodiment 7, the reception data for producing the B mode image are obtained for pertinent sound rays by transmitting the wide ultrasonic beam B2 for producing the B mode image also for the sound rays S4 to S6 on which nine lattice points E in the region of interest R are located without regard to the acquisition of the reception data for measuring the sound speed, whereas, alternatively, the reception data for producing the B mode image may be obtained by using, as the reception data for producing the B mode image, the reception data for measuring the sound speed obtained by forming the transmission focus at the lattice points E having a depth closest to the given depth L at which the B mode image is produced among the lattice points E inside the region of interest R and by transmitting and receiving the ultrasonic beam instead of transmitting the ultrasonic beam B2 for producing the B mode image for the sound rays S4 to S6 passing through the region of interest R to enable the image producer 15 to produce the B mode image.

In brief, in FIG. 14, the reception data for measuring the sound speed obtained for three lattice points E located at the deepest position closest to the given depth L among the nine lattice points E in the region of interest R may be used as reception data for producing the B mode image.

In this case, there may be provided a given depth L1 for producing the B mode image so that the given depth L1 is equal to the depth of any of the lattice points set inside the region of interest R.

As illustrated in FIG. 15, for example, nine lattice points are set inside the region of interest R, and among these lattice points, a total of six lattice points located at the shallowest position and the deepest position become lattice points E1 used exclusively for measuring the sound speed whereas three lattice points located at the center of the region of interest R in the depth direction become lattice points E2 for both the production of the B mode image and the measurement of the sound speed. In FIG. 4, the lattice points E1 are indicated by “”, and the lattice points E2 are indicated by “◯”.

At the depth of the lattice points E2 located at the center of the region of interest R in the depth direction, there is provided the given depth L1 for producing the B mode image.

Then, the transmission focus is sequentially formed for the nine lattice points E1 and E2 inside the region of interest R, and the ultrasonic beam is transmitted and received to obtain the reception data for measuring the sound speed.

The reception data for measuring the sound speed are sequentially outputted from the reception circuit 3 to the signal processor 4 and to the memory 10 for storage, whereupon the sound speed map producer 11 calculates the local sound speeds at the lattice points E1 and E2 and produces the sound speed map for the inside of the region of interest R using the reception data for measuring the sound speed stored in the memory 10.

After the ultrasonic beam B2 having the narrow width portion extending over the three sound rays S1 to S3 at the given depth L is transmitted and the reception data for producing the B mode image for the sound rays S1 to S3 are thereby obtained, the ultrasonic beam B2 having the narrow width portion extending over the three sound rays S7 to S9 at the given depth L is transmitted to obtain the reception data for producing the B mode image for the sound rays S7 to S9. The ultrasonic beam B2 is not transmitted for the sound rays S4 to S6 passing through the region of interest R.

The reception data for producing the B mode image are outputted sequentially from the reception circuit 3 to the signal processor 4 of the image producer 15 and stored in the memory 10.

According to an instruction by the controller 12, the signal processor 4 uses reception data obtained by forming the transmission focus at three points E2 inside the region of interest R among the reception data sequentially entered from the reception circuit 3 and by transmitting and receiving the ultrasonic beam B1 and reception data obtained by transmitting and receiving the wide ultrasonic beam B2 to produce the B mode image signal. Specifically, the reception data for measuring the sound speed obtained by transmitting and receiving the ultrasonic beam B1 with the transmission focus formed at the three lattice points E2 inside the region of interest R are used as reception data for producing the B mode image for the sound rays S4 to S6 and, based on these reception data for producing the B mode image and the reception data for producing the B mode image for the sound rays S1 to S3 and S7 to S9 obtained by transmitting and receiving the wide ultrasonic beam B2, the B mode image signal is produced. The B mode image signal is allowed to undergo raster conversion by the DSC 5 and various image processing by the image processor 6 and sent to the display controller 7.

Upon production of the sound speed map for the region of interest R by the sound speed map producer 11, the sound speed map data are allowed to undergo raster conversion by the DSC 5 and various image processing by the image processor 6 before being transmitted to the display controller 7. Then, according to the display mode entered from the operating unit 13 by the operator, the B mode image is displayed, with the sound speed map superimposed over it, on the monitor 8, or the B mode image and the sound speed map are displayed in juxtaposition on the monitor 8.

In Embodiment 8, by using the reception data for measuring the sound speed obtained by transmitting and receiving the ultrasonic beam B1 with the transmission focus formed at the three lattice points E2 inside the region of interest R also as reception data for producing the B mode image, the number of times the wide ultrasonic beam B2 needs to be transmitted and received can be accordingly reduced, and the reception data for producing the B mode image and the reception data for measuring the sound speed can be more efficiently obtained to achieve both the production of the B mode image and the production of the sound speed map.

Because of the need to calculate the respective local sound speeds at the lattice points in the region of interest R, there are cases where lattice points are added outside the region of interest R, and the transmission focus for measuring the sound speed is formed also at these external lattice points as illustrated in FIG. 16. Also in this case, the reception data for producing the B mode image may be obtained by sequentially forming the transmission focus at lattice points and transmitting and receiving the ultrasonic beam B1 to obtain the reception data for measuring the sound speed while the reception data for producing the B mode image may be obtained by transmitting and receiving the wide ultrasonic beam B2 having the narrow width portion extending over a plurality of sound rays at the given depth L for every two or more sound rays.

In FIG. 16, among the sound rays S1 to S13, the sound rays S6 to S8 are shown as passing through the region of interest R, and the sound rays S1 to S5 and S9 to S13 are shown as passing outside the region of interest R. Also on the sound rays S4, S5, S9, and S10 are set the lattice points E1 as shown.

The transmission focus is sequentially formed at all the lattice points E1 and E2, and the ultrasonic beam B1 is transmitted and received to obtain the reception data for measuring the sound speed, while the reception data for producing the B mode image is obtained by transmitting and receiving the wide ultrasonic beam B2 for the sound rays S1 to S5 and S9 to S13 passing outside the region of interest R.

The reception data for measuring the sound speed obtained by transmitting and receiving the ultrasonic beam B1 with the transmission focus formed at the three lattice points E2 inside the region of interest R are used as reception data for producing the B mode image for the sound rays S6 to S8, so that the B mode image signal is produced based on these reception data for producing the B mode image and the reception data for producing the B mode image for the sound rays S1 to S5 and S9 to S13 obtained by transmitting and receiving the wide ultrasonic beam B2.

While, in Embodiments 1 to 8, the reception data outputted from the reception circuit 3 are first stored in the memory 10, so that the sound speed map producer 11 uses the reception data stored in the memory 10 to calculate the local sound speeds at the lattice points in the region of interest R and produce the sound speed map for the inside of the region of interest R, the sound speed map producer 11 may directly receive the reception data outputted from the reception circuit 3 to produce the sound speed map.

Because the memory 10 stores not only the reception data used for measuring the sound speed but the reception data for producing the B mode image, the reception data for producing the B mode image may be read from the memory 10 as necessary by the control given by the controller 12 for the image producer 15 to produce the B mode image.

While, in Embodiments 1 to 8, the number of apertures, i.e., the number of sound rays in the transducer array 1, and the number of lattice points in the region of interest R as shown, for example, are small for simplicity, the invention is not limited thereto, and the number of apertures and the number of lattice points are preferably determined as appropriate for diagnosis using the B mode image and for measuring the sound speed.

What is claimed is:

1. An ultrasound diagnostic apparatus comprising:
an ultrasound probe including a transducer array;
a transmission circuit for supplying an actuation signal to the transducer array of the ultrasound probe to cause the transducer array to emit ultrasonic beams to a subject;
a reception circuit for processing reception signals outputted from the transducer array of the ultrasonic probe having received ultrasonic echoes from the subject;
a region-of-interest setting unit for setting a region of interest in an imaged region;
a controller for controlling the transmission circuit and the reception circuit to obtain reception data for measuring a sound speed by transmitting and receiving ultrasonic beams with forming transmission focuses at a plurality of points located in a predetermined pattern on sound rays passing inside the region of interest set by the region-of-interest setting unit, and to obtain first reception data for producing a B mode image by transmitting and receiving ultrasonic beams with forming a transmission focus at at least one given depth for sound rays passing outside the region of interest;
a sound speed map producer for producing a sound speed map for the inside of the region of interest based on the reception data for measuring a sound speed; and
an image producer for producing a B mode image based on the first reception data for producing a B mode image and second reception data for producing a B mode image, the second reception data for producing a B mode image being reception data for measuring a sound speed obtained by transmitting and receiving ultrasonic beams with forming transmission focuses at points having a depth closest to the given depth among the plurality of points located in the predetermined pattern on the sound rays passing inside the region of interest.

2. The ultrasound diagnostic apparatus according to claim 1, wherein the controller sets the given depth so as to be equal to that of any one of said plurality of points.

3. The ultrasound diagnostic apparatus according to claim 2, wherein the controller sets the given depth so as to be equal to that of any one of said points located at a center of the region of interest in a depth direction among said plurality of points.

4. The ultrasound diagnostic apparatus according to claim 1, wherein the controller sets a plurality of the given depths for the sound rays passing outside the region of interest

5. The ultrasound diagnostic apparatus according to claim 4, wherein the controller sets two of the given depths that are equal to that of points located in the shallowest position and points located in the deepest position among said plurality of points.

6. An ultrasound diagnostic apparatus comprising:
an ultrasound probe including a transducer array;
a transmission circuit for supplying an actuation signal to the transducer array of the ultrasound probe to cause the transducer array to emit ultrasonic beams to a subject;
a reception circuit for processing reception signals outputted from the transducer array of the ultrasonic probe having received ultrasonic echoes from the subject;
a region-of-interest setting unit for setting a region of interest in an imaged region;
a controller for controlling the transmission circuit and the reception circuit to obtain reception data for measuring a sound speed by transmitting and receiving ultrasonic beams with forming transmission focuses at a plurality of points located in a predetermined pattern inside the region of interest set by the region-of-interest setting unit, and to obtain reception data for producing a B mode image by transmitting and receiving ultrasonic beams with forming a transmission focus at a given depth for each of sound rays;
a sound speed map producer for producing a sound speed map for the inside of the region of interest based on the reception data for measuring a sound speed; and
an image producer for producing a B mode image based on the reception data for producing a B mode image,
the controller controlling the transmission circuit and the reception circuit to obtain the reception data for producing a B mode image during production of the sound speed map by the sound speed map producer after obtaining the reception data for measuring a sound speed.

7. The ultrasound diagnostic apparatus according to claim 6,
wherein the controller controls the transmission circuit and the reception circuit to obtain the reception data for producing a B mode image for sound rays passing outside the region of interest, and
the image producer produces a B mode image by using as reception data for producing a B mode image the reception data for measuring a sound speed obtained by transmitting and receiving ultrasonic beams with forming transmission focuses at points having a depth closest to the given depth among said plurality of points for sound rays passing through the region of interest.

8. The ultrasound diagnostic apparatus according to claim 7, wherein the controller sets the given depth so as to be equal to that of any one of said plurality of points.

9. The ultrasound diagnostic apparatus according to claim 8, wherein the controller sets the given depth so as to be equal to that of any one of said points located at a center of the region of interest in a depth direction among said plurality of points.

10. An ultrasound diagnostic apparatus comprising:
an ultrasound probe including a transducer array,
a transmission circuit for supplying an actuation signal to the transducer array of the ultrasound probe to cause the transducer array to emit ultrasonic beams to a subject,
a reception circuit for processing reception signals outputted from the transducer array of the ultrasonic probe having received ultrasonic echoes from the subject,
a region-of-interest setting unit for setting a region of interest in an imaged region,
a controller for controlling the transmission circuit and the reception circuit to obtain reception data for measuring a sound speed by transmitting and receiving ultrasonic beams with forming transmission focuses at a plurality of points located in a predetermined pattern inside the region of interest set by the region-of-interest setting unit, and to obtain reception data for producing a B mode image by transmitting and receiving an ultrasonic beam having a narrow width portion extending over a plurality of sound rays at a given depth for every two or more sound rays;
a sound speed map producer for producing a sound speed map for the inside of the region of interest based on the reception data for measuring a sound speed; and
an image producer for producing a B mode image based on the reception data for producing a B mode image,

11. The ultrasound diagnostic apparatus according to claim 10,
wherein the controller controls the transmission circuit and the reception circuit to obtain the reception data for producing a B mode image for sound rays passing outside the region of interest, and
the image producer produces a B mode image by using as reception data for producing a B mode image the reception data for measuring a sound speed obtained by transmitting and receiving ultrasonic beams with forming transmission focuses at points having a depth closest to the given depth among said plurality of points for sound rays passing through the region of interest.

12. The ultrasound diagnostic apparatus according to claim 11, wherein the controller sets the given depth so as to be equal to that of any one of said plurality of points.

13. The ultrasound diagnostic apparatus according to claim 12, wherein the controller sets the given depth so as to be equal to that of any one of said points located at a center of the region of interest in a depth direction among said plurality of points.

14. A method of producing an ultrasound image comprising the steps of:
setting a region of interest in an imaged region and setting a plurality of points located in a predetermined pattern on sound rays passing inside the region of interest;
obtaining reception data for measuring a sound speed by transmitting and receiving ultrasonic beams with forming transmission focuses at the plurality of points;
producing a sound speed map for the inside of the region of interest based on the reception data for measuring a sound speed;
obtaining reception data for producing a B mode image by transmitting and receiving ultrasonic beams with forming transmission focuses at at least one given depth for sound rays passing outside the region of interest; and
producing a B mode image based on the first reception data for producing a B mode image and second reception data for producing a B mode image, the second reception data for producing a B mode image being reception data for measuring a sound speed obtained by transmitting and receiving ultrasonic beams with forming transmission focuses at points having a depth closest to the given depth among the plurality of points located in the predetermined pattern on the sound rays passing inside the region of interest.

15. A method of producing an ultrasound image comprising the steps of:
setting a region of interest in an imaged region and setting a plurality of points located in a predetermined pattern inside the region of interest;
obtaining reception data for measuring a sound speed by transmitting and receiving ultrasonic beams with forming transmission focuses at the plurality of points;
starting to produce a sound speed map for the inside of the region of interest based on the reception data for measuring a sound speed;
obtaining reception data for producing a B mode image by transmitting and receiving ultrasonic beams with forming transmission focuses at one given depth during producing of the sound speed map; and
producing a B mode image based on the obtained reception data for producing a B mode image.

16. A method of producing an ultrasound image comprising the steps of:
setting a region of interest in an imaged region and setting a plurality of points located in a predetermined pattern inside the region of interest;
obtaining reception data for measuring a sound speed by transmitting and receiving ultrasonic beams with forming transmission focuses at the plurality of points;
producing a sound speed map for the inside of the region of interest based on the reception data for measuring a sound speed;
obtaining reception data for producing a B mode image by transmitting and receiving an ultrasonic beam having a narrow width portion extending over a plurality of sound rays at a given depth for every two or more sound rays; and
producing a B mode image based on the reception data for producing a B mode image.
